(19) United States
(12) Patent Application Publication
(10) Pub. No.: US 2010/0256576 A1
(43) Pub. Date: Oct. 7, 2010

(54) Disinfectant Compositions, Methods and Systems

(75) Inventors: Dinesh Aggarwal, Franklin, MA (US); Chirag B. Shah, North Attleboro, MA (US)

Correspondence Address: TYCO HEALTHCARE GROUP LP, 15 HAMPSHIRE STREET, MANSFIELD, MA, 02048, US

(73) Assignee: TYCO HEALTHCARE GROUP LP, Mansfield, MA (US)

(21) Appl. No.: 12/755,550

(22) Filed: Apr. 7, 2010

Related U.S. Application Data
(60) Provisional application No. 61/167,415, filed on Apr. 7, 2009.

(51) Int. Cl. A61L 29/16; A61K 31/235; A61P 33/02; A61P 31/04; A61P 31/12; A61P 33/00; A61P 31/00

(52) U.S. Cl. 604/265; 514/544

(57) ABSTRACT

Disinfectant compositions including EDTA salt(s), Parabens and Methylene Blue. The disinfectant compositions have demonstrated activity as enhanced, fast acting catheter lock/flush solutions. They are safe for human and medical uses and may be used as prophylactic preparations to prevent infection, or to reduce the proliferation of and/or eliminate existing or established infections. In addition, the composition has anticoagulant activity and can be utilized to maintain the patency of catheters.

### PRIORITY

The present application claims priority to U.S. provisional application No. 61/167,415 filed on Apr. 7, 2009, the contents of which are expressly incorporated by reference herein.

### BACKGROUND

Infections are a significant problem in many fields where sanitary conditions are important, such as in healthcare. Problematic infections may arise from bacterial, fungal, amoebic, protozoan and/or viral organisms. Challenges are encountered both in preventing infection, and in reducing or eliminating the infection once it is established. Infected environments may include surfaces of objects, fluids and fluid conduits and/or humans or animals.

Alcohol solutions and isopropyl alcohol wipes are commonly used to disinfect surfaces and have been shown to have antibacterial activity. The most effective inhibitory anti-microbial effect is seen with 70% isopropanol solutions. Alcohol solutions at this concentration are quite expensive and rapidly evaporate, which substantially diminishes their efficacy and increases their cost. Moreover, although isopropanol solutions may be used for surfaces, including human skin, and in a variety of medical applications, alcohol solutions of this concentration cannot be administered to humans, for medical purposes, other than topically.

In the healthcare field, infections of various types and causes are common and often result in longer hospital stays, producing higher hospital costs. Even worse, over 90,000 patient deaths annually are attributed to nosocomial infections—that is, infections acquired at a hospital or in another healthcare environment. Surveillance for nosocomial infection has become an integral part of hospital practice. Studies conducted more than 20 years ago by the Centers for Disease Control and Prevention (CDC) documented the efficacy of these surveillance activities in reducing nosocomial infection occurrence. Despite the attention paid to problems of nosocomial infection, however, infection rates have not been dramatically reduced, and nosocomial infections remain a substantial risk and a substantial health concern.

One problematic source of infections in the medical and veterinary fields is found in catheters, and particularly in in-dwelling catheters. Catheters have become essential in the management of critical care patients, yet the inside of a catheter is often the major source of infection. Catheters are used for delivery of fluids, blood products, drugs, nutrients, hemodialysis, hemofiltration, peritoneal dialysis, retrieval of blood samples, monitoring of patient conditions, etc. Transcutaneous catheters often become infected through skin penetration of the catheter. It has been found that seventy percent (70%) of all nosocomial bloodstream infections occur in patients with central venous catheters. Daouicher et al. 340, 1-8, NEW ENGLAND JOURNAL OF MEDICINE (1999).

In particular, during some procedures, a catheter must be implanted in, and remain implanted in, a patient for a relatively long period of time, e.g. over thirty days. Intravenous (IV) therapy catheters and urinary catheters typically remain implanted for a substantial period of time. As a result of trauma to the areas of insertion, and pain to the patients, such catheters can't be removed and implanted frequently. Catheter-borne bacteria are implicated as a primary source of urinary tract infections. Patients who receive a peripherally inserted central catheter during pregnancy have also been found to be at significant risk for infectious complications. “Complications Associated With Peripherally Inserted Central Catheter Use During Pregnancy” AM. J. OBSTET. GYCOL. 188(5):1223-5 May 2003. In addition, central venous catheter infection, resulting in catheter related sepsis, has been cited as the most frequent complication during home parenteral nutrition. CLINICAL NUTRITION, 21(1):33-38, 2002. Because of the risk of infections, catheterization may be limited to incidences when the procedure is absolutely necessary. This seriously compromises patient health.

After most prescribed access medical procedures involving a catheter, the catheter is flushed with saline and then filled with a liquid, such as saline or a heparin solution, to prevent blood from clotting inside of the catheter, to inhibit the patient's blood from backing up into the catheter, and to prevent gases from entering the catheter. The liquid that is used to flush the catheter is referred to as a “lock-flush,” and the liquid used to fill the catheter following flushing or during periods of non-use is referred to as a “lock” solution.

Traditionally, catheters have been locked with normal saline or heparin solutions. Heparin and saline are sometimes used in combination. Normal saline is generally used to lock short term peripheral intravenous catheters, but saline has no anticoagulant or antimicrobial activity. Heparin solutions are generally used to lock vascular catheters. Heparin has anticoagulant activity but it does not function as an antimicrobial and does not prevent or ameliorate infections. There are also strong indications that heparin in lock solutions may contribute to heparin-induced thrombocytopenia, a serious bleeding complication that occurs in a subset of patients receiving heparin injections.

Catheter locking solutions comprising taurolidine, citric acid and sodium citrate have been proposed. A recent publication (Kidney International, September 2002) describes the use of a 70% alcohol solution as a lock solution for a subcutaneous catheter port. The use of alcohol as a lock solution is questionable, since it is generally not considered to be an anticoagulant, and since there would be risks associated with this solution entering the bloodstream. There is also no evidence that the inventors are aware of that indicates that a 70% alcohol solution has any biofilm eradication activity.

An emerging trend and recommendation from the Center for Infectious Disease (CID) is to treat existing catheter infections systemically with either a specific or a broad range antibiotic. Use of an antibiotic in a lock solution to prevent infection is not recommended. The use of antibiotics to treat existing catheter infections has certain risks, including: (1) the risk of antibiotic-resistant strains developing; (2) the inability of the antibiotic to kill sessile, or deep-layer biofilm bacteria, which may require the use of antibiotics at toxic concentrations; and (3) the high cost of prolonged antibiotic therapy. Catheters coated with a disinfectant or antibiotic material are available. These coated catheters may only provide limited protection for a relatively short period of time.

In general, free-floating organisms may be vulnerable to antibiotics. However, bacteria and fungi may become impervious to antibiotics by attaching to surfaces and producing a slimy protective substance, often referred to as extra-cellular polymeric substance (EPS), polysaccharide covering or glycocalyx. As the microbes proliferate, more than 50 genetic up or down regulations may occur, resulting in the formation of a more antibiotic resistant microbial biofilm. One article attributes two-thirds of the bacterial infections that physicians encounter to biofilms. SCIENCE NEWS, 1-5 Jul. 14, 2001.

Biofilm formation is a genetically controlled process in the life cycle of bacteria that produces numerous changes in the cellular physiology of the organism, often including increased antibiotic resistance (of up to 100 to 1000 times), as compared to growth under planktonic (free floating) conditions. As the organisms grow, problems with overcrowding and diminishing nutrition trigger shedding of the organisms to seek new locations and resources. The newly shed organisms quickly revert back to their original free-floating phase and are once again vulnerable to antibiotics. However, the free-floating organism may enter the bloodstream of the patient, creating bloodstream infections, which produce clinical signs, e.g. fever, and more serious infection-related symptoms. Sessile rafts of biofilm may slough off and may attach to tissue surfaces, such heart valves, causing proliferation of biofilm and serious problems, such as endocarditis.

To further complicate matters, conventional sensitivity tests measure only the antibiotic sensitivity of the free-floating organisms, rather than organisms in a biofilm state. As a result, a dose of antibiotics is administered to the patient, such as through a catheter, in amounts that rarely have the desired effect on the biofilm phase organisms that may reside in the catheter. The biofilm organisms may continue to shed more planktonic organisms or may go dormant and proliferate later as an apparent recurrent infection.

In order to eradicate biofilm organisms through the use of antibiotics, a laboratory must determine the concentration of antibiotic required to kill the specific genetic biofilm phase of the organism. Highly specialized equipment is required to provide the minimum biofilm eradication concentration. Moreover, the current diagnostic protocols are time consuming, and results are often not available for many days, e.g. five (5) days. This time period clearly doesn't allow for prompt treatment of infections. The delay and the well-justified fear of infection may result in the overuse of broad-spectrum antibiotics and continued unnecessary catheter removal and replacement procedures. Overuse of broad-spectrum antibiotics can result in the development of antibiotic resistant bacterial strains, which cannot be effectively treated. Unnecessary catheter removal and replacement is painful, costly and may result in trauma and damage to the tissue at the catheter insertion site.

The antibiotic resistance of biofilms, coupled with complications of antibiotic use, such as the risk of antibiotic resistant strains developing, has made antibiotic treatment an unattractive option. As a result, antibiotic use is limited to symptomatic infections and prophylactic antibiotics are not typically applied to prevent contamination. Because the biofilm can act as a selective phenotypic resistance barrier to most antibiotics, the catheter must often be removed in order to eradicate a catheter related infection. Removal and replacement of the catheter is time consuming, stressful to the patient, and complicates the medical procedure. Therefore, there are attempts to provide convenient and effective methods for killing organisms, and especially those dwelling inside of catheters, without the necessity of removing the catheter from the body.

In addition to bacterial and fungal infections, amoebic infections can be very serious and painful, as well as potentially life threatening. Several species of Acanthamoeba, for example, have been found to infect humans. Acanthamoeba are found worldwide in soil and dust, and in fresh water sources as well as in brackish water and sea water. They are frequently found in heating, venting and air conditioner units, humidifiers, dialysis units, and in contact lens paraphernalia. Acanthamoeba infections, in addition to microbial and fungal infections, may also be common in connection with other medical and dental devices, including toothbrushes, dentures and other dental appliances, and the like. Acanthamoeba infections often result from improper storage, handling and disinfection of contact lenses and other medical devices that come into contact with the human body, where they may enter the skin through a cut, wound, the nostrils, the eye, and the like.

There is a need for improved methods and substances to prevent and destroy infections in catheters. Such disinfectant solutions should have a broad range of antimicrobial properties. In particular, the solutions should be capable of penetrating biofilms to eradicate the organisms comprising the biofilms. The methods and solutions should be safe enough to be use as a preventive measure as well as in the treatment of existing infections.

### OBJECTS AND SUMMARY

Parabens are a group of chemicals widely used as preservatives in the cosmetic and pharmaceutical industries. Parabens are effective preservatives in many types of formulas. They can be found in shampoos, commercial moisturizers, shaving gels, personal lubricants, topical/parenteral pharmaceuticals, spray tanning solution and toothpaste. They are also used as food additives.

Methylene blue (MB) is a heterocyclic aromatic chemical compound with molecular formula: C₁₆H₁₈ClN₃S. At room temperature it appears as a solid, odorless, dark green powder, that yields a blue solution when dissolved in water. The hydrated form has 3 molecules of water per molecule of MB. Methylene blue should not be confused with methyl blue, another histology stain, new methylene blue, nor with the methyl violets often used as pH indicators.

Ethylene diamine tetraacetic acid, and salts thereof, (EDTA) has been used for systemic detoxification treatment and as an anticoagulant in blood samples for some time. Thus, its use for medical treatment and applications is established. The use of disodium EDTA and calcium disodium EDTA in combination with other compounds to enhance anti-microbial properties of these other compounds has been studied and practiced. It has been discovered that many stand-alone salts of Ethylenediamine-tetraaceticacid (EDTA) are effective anti-microbial agents and that specific salts are more effective than others. In particular, it has been discovered that certain salts of EDTA exhibit anti-microbial (both antifungal and antibacterial) properties superior to those of the disodium salt in common use. In particular, dipotassium and ammonium EDTA are superior to disodium EDTA, and tetrasodium EDTA (TEDTA) has been found to be preferred to disodium, ammonium, and dipotassium.

In the following discussion, the terms “microbe” or “microbial” will be used to refer to microscopic organisms or matter, including fungal and bacterial organisms, and possibly including viral organisms, capable of infecting humans. The term “anti-microbial” will thus be used herein to refer to a material or agent that kills or otherwise inhibits the growth of fungal and/or bacterial and possibly viral organisms.

The term “disinfect” will be used to refer to the reduction, inhibition, or elimination of infectious microbes from a defined system. The term “disinfectant” will be used herein to refer to a one or more anti-microbial substances used either alone or in combination with other materials such as carriers, solvents, or the like.

The term “bactericidal activity” is used to refer to an activity that at least essentially kills an entire population of bacteria, instead of simply just reducing or inhibiting their growth. The term “fungicidal activity” is used to refer to an activity that at least essentially kills an entire population of yeast, instead of simply just reducing or inhibiting their growth. Contamination of conduits, e.g., catheters, poses serious and substantial health risks and bactericidal disinfection is a significant priority.

The term “infected system” will be used herein to refer to a defined or discrete system or environment in which one or more infectious microbes are or are likely to be present. Examples of infected systems include a physical object such a medical device, a biological system such as the human body, or a combination of a physical object and a biological system such as a catheter or the like arranged at least partly within a human body. Tubes and other conduits for the delivery of fluids, in healthcare settings, may also define an infected system.

A solution that consists essentially of EDTA salt(s), Parabens and Methylene Blue in a solvent, preferably an aqueous based solvent such as water, saline or PBS, is substantially free from other active substances having antimicrobial and/or anti-fungal activity.

The present disclosure involves disinfectant solutions comprising, or consisting essentially of, or consisting of, EDTA salt(s), Parabens and Methylene Blue at a prescribed concentration and/or pH. The inventors have discovered, unexpectedly, that certain EDTA salt(s), Parabens and Methylene Blue formulations provide enhanced disinfectant activities. EDTA salt(s), Parabens and Methylene Blue formulations act as enhanced, fast acting catheter lock/flush solutions. EDTA salt(s), Parabens and Methylene Blue formulations of the present disclosure are also highly effective in killing pathogenic biofilm organisms, and are expected to be effective in reducing existing biofilms, in eliminating existing biofilms as well as preventing biofilm formation. EDTA salt(s), Parabens and Methylene Blue formulations function as broad-spectrum anti-microbial agents, as well as fungicidal agents. EDTA salt(s), Parabens and Methylene Blue formulations are expected to exhibit anti-protozoan activity and also exhibit anti-amoebic activity.

The EDTA salt(s), Parabens and Methylene Blue formulations of the present disclosure are expected to be safe for human administration and are biocompatible and non-corrosive. The disinfectant solutions of the present disclosure have applications at least as lock and lock flush solutions for various types of catheters. The efficacy of the EDTA salt(s), Parabens and Methylene Blue formulations of the present disclosure is superior to many disinfectant compositions conventionally used as catheter lock/flush solutions. Preferred embodiments of the disclosed formulations do not contribute to antibiotic resistance, which provides yet another important benefit.

The EDTA salt(s), Parabens and Methylene Blue formulations of the present disclosure are also have improved anticoagulant properties and are thus especially useful as catheter lock-flush solutions and other related uses.

In one embodiment, disinfectant compositions of the present disclosure have some of the following properties: anticoagulant properties; inhibitory and/or bactericidal activity against a broad spectrum of bacteria in a planktonic form; inhibitory and/or fungicidal activity against a spectrum of fungal pathogens; inhibitory and/or bactericidal activity against a broad spectrum of bacteria in a sessile form; inhibitory activity against protozoan infections; inhibitory activity against Acanthamoeba infections; safe and biocompatible, at least in modest volumes, in contact with a patient; and safe and biocompatible, at least in modest volumes, in a patient's bloodstream.

Methods for inhibiting the growth and proliferation of microbial populations and/or fungal pathogens are provided that comprise contacting an infected system, such as an infected or suspected infected object, or surface, e.g., catheter, with a disinfectant composition of the present disclosure. Methods for inhibiting the growth and proliferation of protozoan populations are also provided, comprising contacting an infected system, such as an infected or suspected infected object, or surface, e.g., catheter, with a disinfectant composition of the present disclosure.

Embodiments of the disclosure include methods for inhibiting the growth and proliferation of amoebic populations, and for preventing amoebic infection, particularly Acanthamoeba infections, comprising contacting an infected system, such as an infected or suspected infected object, or a surface, e.g., catheter, with a disinfectant composition of the present disclosure wherein efficacy is expected.

Embodiments of the disclosure include methods for substantially eradicating microbial populations and comprise contacting an infected system, such as an infected or suspected infected object, or surface, e.g., catheter, with a disinfectant composition of the present disclosure wherein efficacy is expected. Embodiments of the disclosure include methods for substantially eradicating an Acanthamoeba population and comprise contacting an infected system, such as an infected or suspected infected object, or surface, e.g., catheter, with a disinfectant composition of the present disclosure wherein efficacy is expected. Depending on the disinfectant composition used in the various methods, various compositions and contact time periods may be required to inhibit the formation and proliferation of various populations, and/or to substantially eradicate various populations. Suitable contact time periods for various compositions may be determined by routine experimentation.

Importantly, in most embodiments, disinfectant compositions and methods of the present disclosure do not employ traditional antibiotic agents and thus do not to contribute to the development of antibiotic resistant organisms.

In one embodiment, disinfectant compositions consisting of, consisting essentially of, or comprising EDTA salt(s), Parabens and Methylene Blue are provided as disinfectant compositions of the present disclosure. Such disinfectant compositions have application as lock solutions and lock flush solutions for various types of in-dwelling access catheters, including vascular catheters used for delivery of fluids, blood products, drugs, nutrition, withdrawal of fluids or blood, dialysis, monitoring of patient conditions, and the like. Disinfectant solutions of the present disclosure may also be used as lock and lock flush solutions for urinary catheters, nasal tubes, throat tubes, and the like. The general solution parameters described below are suitable for these purposes. In one embodiment, a disinfectant solution consisting of, consisting essentially of, or comprising EDTA salt(s), Parabens and Methylene Blue is provided to maintain the patency of in-dwelling intravascular access devices. Methods for disinfectant catheters and other medical tubes, such as nasal tubes, throat tubes, and the like, are also provided and involve contacting the catheter or other medical tube with a disinfectant composition of the present disclosure. Examples of a catheter include peripherally inserted catheters, central venous catheters, peritoneal catheters, hemodialysis catheters and urological catheters. Disinfectant compositions of the present disclosure are expected to be providable in a dry form which, upon introduction of a suitable solvent, forms a solution.

### BRIEF DESCRIPTION OF THE DRAWING(S)

FIG. 1 shows the results of a Rate of Kill (ROK) experiment with S. Aureus.

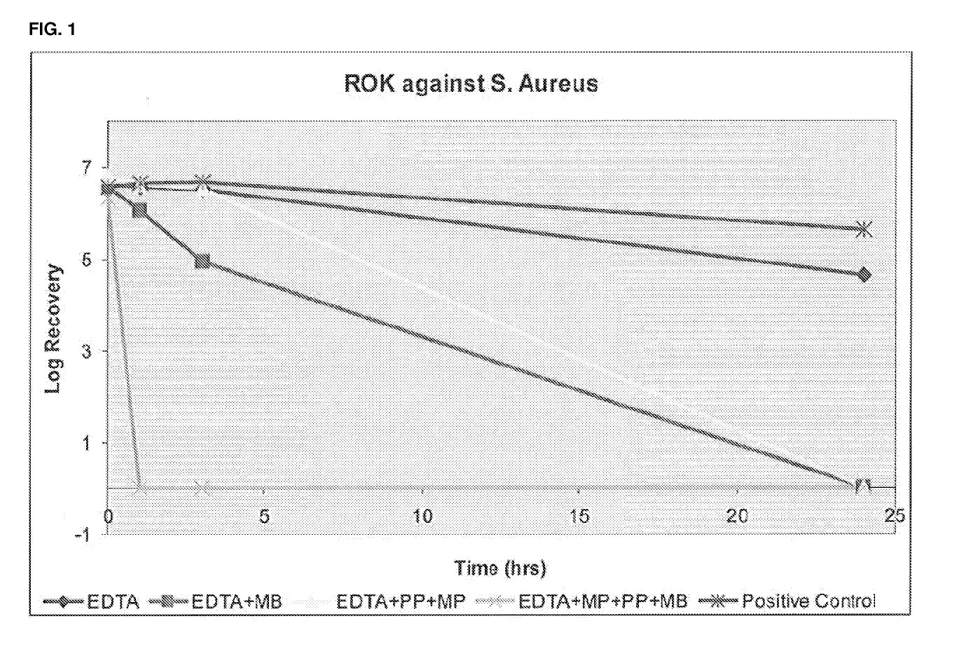

FIG. 2 shows the results of a Rate of Kill (ROK) experiment with S. Epidermidis.

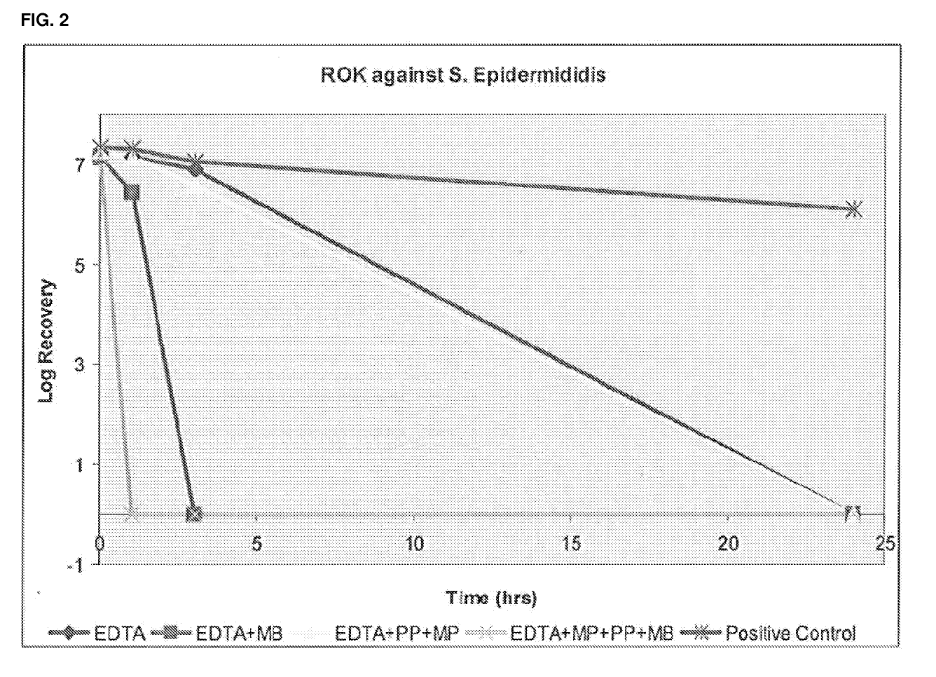

FIG. 3 shows the results of a Rate of Kill (ROK) experiment with C. Albicans.

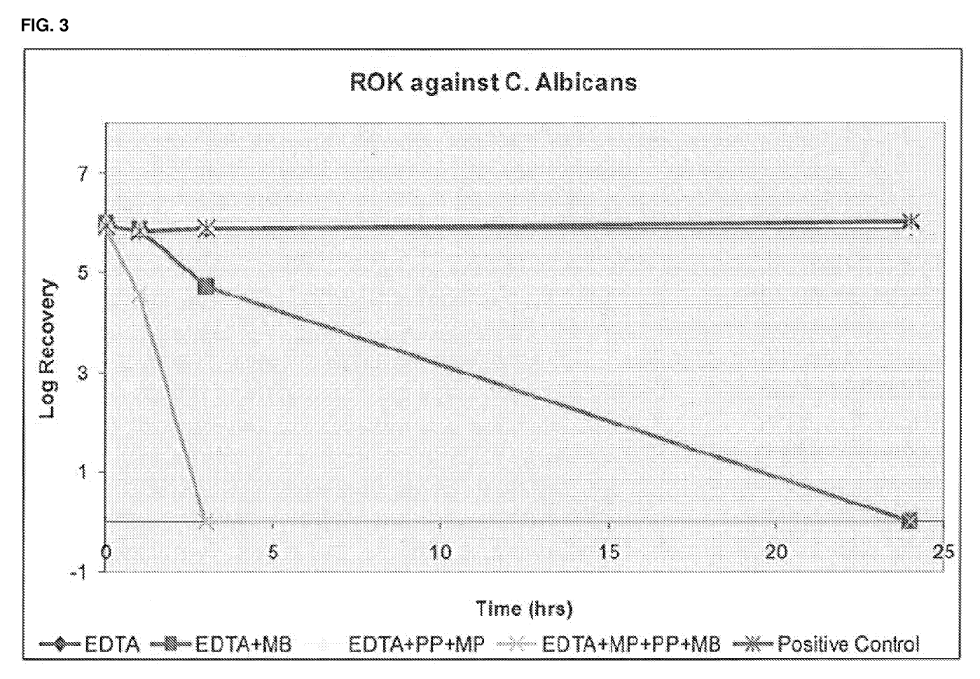

FIG. 4 shows the results of a Rate of Kill (ROK) experiment with P. Aeruginosa.

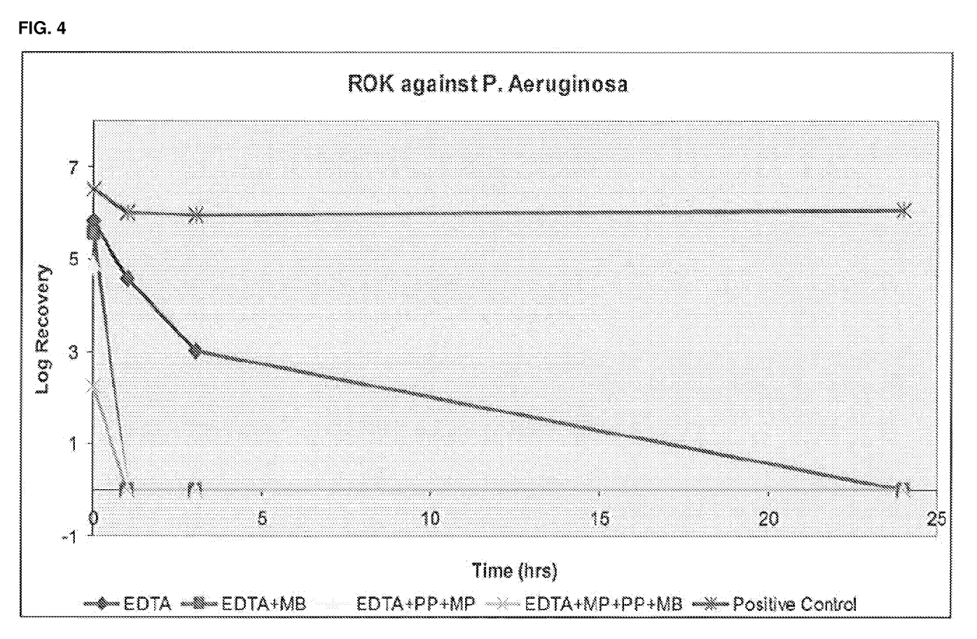

FIG. 5 shows the results of experiments of a Prothrombin Time (PT) Assay.

FIG. 6 shows the graph of the International Normalized Ratio (INR) for EDTA, MB, EDTA+MB, EDTA+PP+MP & EDTA+PP+MP+MB formulation from a Prothrombin Time (PT) Assay.

FIG. 7 shows the percentage relationship of species of an EDTA salt in relation to the pH of the solution.

### DETAILED DESCRIPTION

Disinfectant compositions of the present disclosure may comprise concentrations of EDTA salt(s), Parabens and Methylene Blue. EDTA salt(s), Parabens and Methylene Blue may be used in compositions with water as the solvent.

Parabens are esters of para-hydroxybenzoic acid, from which the name is derived. Common parabens include methylparaben, ethylparaben, propylparaben and butylparaben. Less common parabens include isobutylparaben, isopropylparaben, benzylparaben and their sodium salts. The general chemical structure of a paraben is shown below, where R symbolizes an alkyl group such as methyl, ethyl, propyl or butyl:

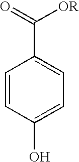

Embodiments of the disclosed composition may include one or more of the following parabens: methylparaben, ethylparaben, propylparaben, butylparaben isobutylparaben, isopropylparaben, benzylparaben and their sodium salts. Preferred parabens are: methylparaben, ethylparaben, propylparaben, benzylparaben and butylparaben. Preferred combinations of parabens are: methylparaben, propylparaben and butyl paraben & ethyl and butyl paraben. Most preferred is a combination of methyl paraben and propyl paraben.

Compositions comprising parabens have a well established safety profile in connection with medical usage and administration to humans. Parabens are also present, in combination with other components, in many solutions used in medical and human health applications, and has been established as safe for human use, both in vitro and in vivo. Parabens is readily available at a reasonable cost, and is stable over time in solution.

Methylene Blue (MB) (also known as Basic Blue 9,trihydrate; Methylene blue trihydrate; 3,7-Bis(dimethylamino)phenazathionium chloride trihydrate, Ehrlich's reagent III, Loeffler's Methylene Blue) is a basic heterocyclic aromatic chemical compound with the following formula:

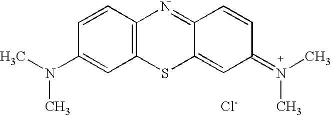

MB has been used in the treatment of malaria, methemoglobinemia, in locating surgical sites, and for validation of properly following medication prescriptions. Long-term use in humans has not been associates with adverse effects in treating methemoglobin toxicities. MB treatment of blood plasma has been reported to affect the functional activity of various coagulation proteins and inhibitors.

Soluble salts of EDTA are used in compositions of the present disclosure. Sodium salts of EDTA are commonly available and generally used, including di-sodium, tri-sodium and tetra-sodium salts, although other EDTA salts, including ammonium, di-ammonium, potassium, di-potassium, cupric di-sodium, magnesium di-sodium, ferric sodium, and combinations thereof, may be used, provided they have the antibacterial and/or fungicidal and/or anti-protozoan and/or anti-amoebic properties desired, and provided that they are sufficiently soluble in the solvent desired. Various combinations of EDTA salts may be used and may be preferred for particular applications.

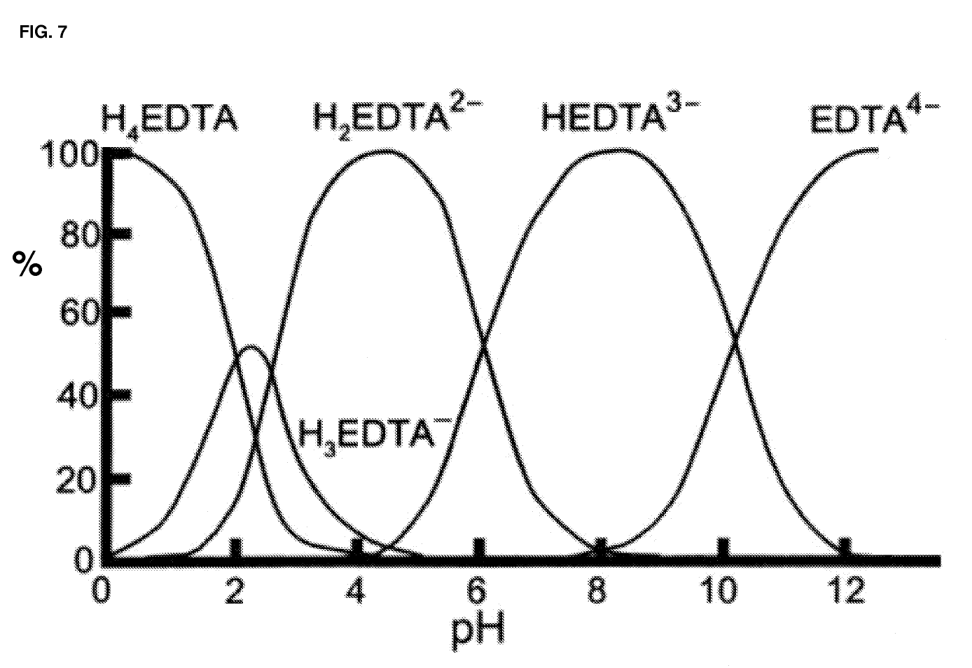

The British Pharmacopoeia (BP) specifies that a 5% solution of di-sodium EDTA has a pH of 4.0 to 5.5. The BP also specifies a pH range of 7.0 to 8.0 for solutions of tri-sodium EDTA. At physiological pH, the sodium salts of EDTA exist as a combination of di-sodium and tri-sodium EDTA, with the tri-sodium salt of EDTA being predominant. In the U.S., pharmaceutical “di-sodium” EDTA prepared for injection has generally been titrated with sodium hydroxide to a pH of 6.5 to 7.5. At this pH, the EDTA solution actually comprises primarily tri-sodium EDTA, with a lesser proportion of the di-sodium salt. FIG. 7 shows the percentage relationship of species of an EDTA salt in relation to the pH of the solution. Other compositions comprising sodium salts of EDTA that are used in medical or healthcare applications are generally adjusted to a pH that is substantially physiological.

Compositions comprising EDTA have a well established safety profile in connection with medical usage and administration to humans. Doses of up to 3000 mg EDTA disodium are infused over 3 hours, on a daily basis, for the treatment of hypercalcemia in humans. This dose is well tolerated. EDTA salts are also present, in combination with other components, in many solutions used in medical and human health applications, and have been established as safe for human use, both in vitro and in vivo. EDTA salts are readily available at a reasonable cost, and are stable over time in solution.

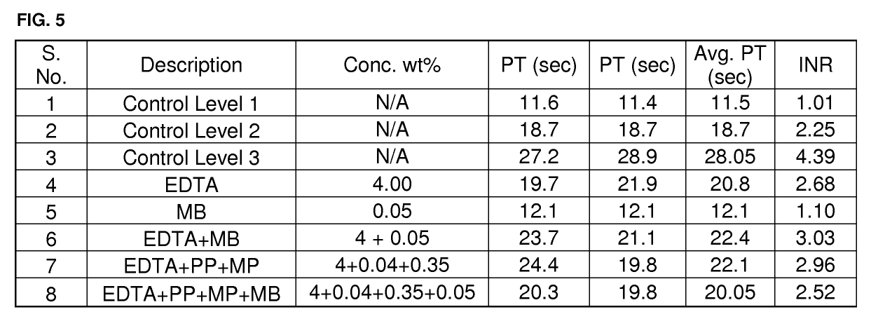

The combination of EDTA salt(s), Parabens and Methylene Blue has an anti-coagulant effect. The anti-coagulant effect is further detailed in FIGS. 5 & 6.

In a further embodiment, a composition of the present disclosure may be minocycline-free, and preferably antibiotic-free. To be minocycline-free or antibiotic-free means to be free of an amount of minocycline or all antibiotics, respectively, that adds a measurable antimicrobial effect. Embodiments of the disclosed composition may also be completely free of minocycline or all antibiotics. Embodiments of the disclosed composition may be free of Polyhexamethylene biguanide (PHMB). To be free of PHMB means to be free of an amount of PHMB that adds a measurable antimicrobial effect. Embodiments of the disclosed composition may also be completely free of Polyhexamethylene biguanide (PHMB).

Embodiments of the disclosed composition may comprise at least 0.005% of one or more parabens, by weight per volume solution (w/v) and less than 1% (w/v) parabens. Embodiments comprising at least 0.01% of one or more parabens and less than 1% (w/v) or less than 0.5% (w/v) of one or more parabens are preferred for many applications, and compositions comprising about 0.01-0.4% (w/v) of one or more parabens are especially preferred.

Embodiments of the disclosed composition may comprise at least 0.001% MB, by weight per volume solution (w/v) and less than 1% (w/v) MB. Embodiments comprising at least 0.01% MB and less than 0.5% (w/v) MB are preferred for many applications, and compositions comprising about 0.05-0.015% (w/v) MB are especially preferred.

Embodiments of the disclosed composition may comprise at least 0.01% EDTA salt(s), by weight per volume solution (w/v) and less than 20.0% (w/v) or less than 16.0% (w/v) EDTA salt(s). Embodiments comprising at least 0.25% (w/v) EDTA salt(s) and less than 12% (w/v) or less than 8% (w/v) EDTA salt(s) are preferred for many applications, and compositions comprising about 0.5-4 (w/v) EDTA salt(s) are especially preferred.

Embodiments of the disclosed composition may comprise between 0 and 25% (v/v) ethanol and water. Other embodiments of the disclosed composition may comprise between 0 and 20% (v/v) ethanol and water, between 0 and 15% (v/v) ethanol and water, or between 0 and 10% (v/v) ethanol and water.

The desired EDTA salt(s), Parabens and Methylene Blue concentrations for various applications may depend on the type of infection being treated and, to some degree, on the solvent used for disinfectant compositions. When aqueous solvents comprising ethanol are used, for example, the concentrations of EDTA salt(s), Parabens and Methylene Blue required to provide the desired level of activity may be reduced compared to the EDTA salt(s), Parabens and Methylene Blue concentrations used in compositions having water as the solvent. “Effective” concentrations of EDTA salt(s), Parabens and Methylene Blue in disinfectant compositions of the present disclosure for inhibitory, bactericidal, fungicidal, biofilm eradication and other purposes, may be determined by routine experimentation.

In certain embodiments, disinfectant compositions of the present disclosure comprise, or consist essentially of, or consist of, EDTA salt(s), Parabens and Methylene Blue in solution at a pH greater than 7.5, and preferably at a pH> or ≧8.0, preferably at a pH of > or ≧8.0 and <or ≦12.0, at a pH> or ≧8.0 and <or ≦11.0, or at a pH> or ≧8.0 and <or ≦10.0, or at a pH> or ≧8.0 and <or ≦9.5 or at a pH> or ≧8.0 and <or ≦9.0, or at a pH of about 8.5. Compositions comprising EDTA salt(s), Parabens and Methylene Blue that are used in medical or healthcare applications may be adjusted to a pH that is substantially physiological. In one embodiment, disinfectant compositions of the present disclosure comprise, or consist essentially of, or consist of, a sodium EDTA salt (or combination of sodium salts), Parabens and Methylene Blue in solution at a pH greater than 7.5, and, in another embodiment, in the range between 8.0 and 12.0 and, in another embodiment, at a pH of between 8.0 and 9.5 and, in yet another embodiment, at a pH of between 8.0 and 9.0. When used herein, the term “EDTA salt” may refer to a single salt, such as a di-sodium or tri-sodium or tetra-sodium salt, or another EDTA salt form, or it may refer to a combination of such salts. The composition of EDTA salt(s) depends both on the EDTA salts used to formulate the composition, and on the pH of the composition. For disinfectant compositions of the present disclosure comprising sodium EDTA salt(s), and at the desired pH ranges (8.0-9.5), the sodium EDTA salts are predominantly present in the tri-sodium salt forms.

Disinfectant compositions comprising, or consisting essentially of, or consisting of EDTA salt(s), Parabens and Methylene Blue have different “effective” pH ranges. “Effective” pH ranges for desired EDTA salt(s) in disinfectant compositions of the present disclosure for inhibitory, bactericidal, fungicidal, biofilm eradication and other purposes, may be determined by routine experimentation.

In some embodiments, disinfectant compositions of the present disclosure consist of EDTA salt(s), Parabens and Methylene Blue, as described above, and disinfectant solutions consist of EDTA salt(s), Parabens and Methylene Blue dissolved in a solvent, generally an aqueous solvent such as water, saline or PBS. In other embodiments, disinfectant compositions of the present disclosure consist essentially of EDTA salt(s), Parabens and Methylene Blue, as described above, generally in an aqueous solvent such as water, saline or PBS.

In some embodiments, disinfectant compositions of the present disclosure comprise EDTA salt(s), Parabens and Methylene Blue having specified concentrations, at specified pH ranges, and may contain materials, including active components, in addition to the EDTA salt(s), Parabens and Methylene Blue described above. Other antimicrobial or biocidal components may be incorporated in disinfectant compositions of the present disclosure comprising EDTA salt(s), Parabens and Methylene Blue, although the use of traditional antibiotics and biocidal agents is generally discouraged as a result of the potential dire consequences of the development of antibiotic- and biocidal-resistant organisms. In some embodiments, disinfectant compositions of the present disclosure comprising EDTA salt(s), Parabens and Methylene Blue having specified concentration(s), at specified pH ranges, are substantially free from other active substances having substantial antimicrobial and/or anti-fungal activity.

Other active and inactive components may also be incorporated in disinfectant compositions of the present disclosure comprising EDTA salt(s), Parabens and Methylene Blue, preferably provided that they don't deleteriously affect the activity and/or stability of the EDTA salt(s), Parabens and Methylene Blue. Proteolytic agents may be incorporated in disinfectant compositions for some applications. Disinfectant compositions formulated for topical application have various creams, emollients, skin care compositions such as aloe vera, and the like, for example. Disinfectant compositions of the present disclosure provided in a solution formulation may also comprise other active and inactive components, preferably provided they don't interfere, deleteriously, with the activity and/or stability of the EDTA salt(s), Parabens and Methylene Blue.

The compositions of the present disclosure may be used in a solution or a dry form. In solution, the EDTA salt(s), Parabens and Methylene Blue are preferably dissolved in a solvent, which may comprise an aqueous solution, such as water or saline, or another biocompatible solution in which the EDTA salt(s), Parabens and Methylene Blue are soluble. Other solvents, including alcohol solutions, may also be used. In one embodiment, EDTA salt(s), Parabens and Methylene Blue compositions of the present disclosure may be formulated in a mixture of water and ethanol. Such solutions are expected to be highly efficacious and may be prepared by making a concentrated EDTA salt(s), Parabens and Methylene Blue stock solution in water and then introducing the desired concentration of ethanol. Ethanol concentrations of from more than about 0.5% and less than about 10%, v/v, are expected to provide effective disinfectant compositions. In some embodiments, bio-compatible non-aqueous solvents may also be employed, provided the EDTA salt(s) can be solubilized and remain in solution during storage and use.

EDTA salt(s), Parabens and Methylene Blue solutions of the present disclosure are preferably provided in a sterile and non-pyrogenic form and may be packaged in any convenient fashion. In some embodiments, disinfectant EDTA salt(s), Parabens and Methylene Blue compositions of the present disclosure may be provided in connection with or as part of a medical device, such as in a pre-filled syringe or another medical device. The compositions may be prepared under sterile, aseptic conditions, or they may be sterilized following preparation and/or packaging using any of a variety of suitable sterilization techniques. Single use vials, syringes or containers of EDTA salt(s), Parabens and Methylene Blue solutions may be provided. Multiple use vials, syringes or containers may also be provided. Systems of the present disclosure include such vials, syringes or containers containing the EDTA salt(s), Parabens and Methylene Blue solutions of the present disclosure. Catheters contemplated for use include peripherally inserted catheters, central venous catheters, peritoneal catheters, hemodialysis catheters and urological catheters.

The compositions of the present disclosure may also be provided in a substantially “dry” form, such as a substantially dry coating on a surface of tubing, or a conduit, or a medical device such as a catheter or conduit, or the like. Dry forms of the disinfectant compositions of the present disclosure may include hydrophilic polymers such as PVP, which tend absorb water and provide lubricity, surfactants to enhance solubility and/or bulking and buffering agents to provide thermal as well as pH stability. Such substantially dry forms of EDTA salt(s), Parabens and Methylene Blue compositions of the present disclosure may be provided in a powder or lyophilized form that may be reconstituted to form a solution with the addition of a solvent. Substantially dry forms of EDTA salt(s), Parabens and Methylene Blue compositions may alternatively be provided as a coating, or may be incorporated in a gel or another type of carrier, or encapsulated or otherwise packaged and provided on a surface as a coating or in a container. Such substantially dry forms of EDTA salt(s), Parabens and Methylene Blue compositions of the present disclosure are formulated such that in the presence of a solution, the substantially dry composition forms an EDTA salt(s), Parabens and Methylene Blue solution having the composition and properties described above. In certain embodiments, different encapsulation or storage techniques may be employed such that effective time release of the EDTA salt(s), Parabens and Methylene Blue is accomplished upon extended exposure to solutions. In this embodiment, the substantially dry EDTA salt(s), Parabens and Methylene Blue solutions may provide disinfectant activity over an extended period of time and/or upon multiple exposures to solutions.

Formulation and production of disinfectant compositions of the present disclosure are generally straightforward. In one embodiment, desired disinfectant compositions of the present disclosure are formulated by dissolving EDTA salt(s), Parabens and Methylene Blue in an aqueous solvent, such as purified water, to the desired concentration and adjusting the pH of the solution to the desired pH. In alternative embodiments, desired disinfectant compositions of the present disclosure are formulated by dissolving EDTA salt(s), Parabens and Methylene Blue in a solvent in which the EDTA salt(s), Parabens and Methylene Blue are soluble to provide a concentrated, solubilized solution, and additional solvents or components may then be added, or the solubilized composition may be formulated in a form other than a solution, such as a topical preparation. The disinfectant solution may then be sterilized using conventional means, such as filtration and/or ultrafiltration, and other means. The preferred osmolarity range for EDTA salt(s), Parabens and Methylene Blue solutions is from 100-500 mOsm/Kg, more preferably from 300-420 mOsm/Kg. The solutions are preferably formulated using USP materials.

A EDTA salt(s), Parabens and Methylene Blue solution can be used as a treatment for catheters defining an infected system. The EDTA salt(s), Parabens and Methylene Blue solution may inhibit microbe colonization by treating the catheter with the solution at the prescribed concentration using a liquid lock prior to and in between infusions and/or by surface coating of catheter devices. A further application is the treatment of colonized or infected catheters by use of a liquid lock containing the EDTA salt(s), Parabens and Methylene Blue solution in the preferred concentration and pH. An EDTA salt(s), Parabens and Methylene Blue solution can be used as an effective decontamination agent for a medical device.

Typically, the EDTA salt(s), Parabens and Methylene Blue solution, when used to treat catheters, are dissolved in water as a carrier, although other carriers may be used. Substances such as thrombolytics, sodium, alcohol, or reagents may also be added to the basic water/EDTA salt(s), Parabens and Methylene Blue solution.

Surprisingly, the combination of EDTA salt(s), Parabens and Methylene Blue is expected to have an unexpectedly enhanced effect against biofilms. Without being held to theory, it is believed that enhanced activity may result from EDTA acting as a chelating agent making microorganisms residing in the biofilm planktonic. Parabens mixed with Methylene Blue may then act effectively against susceptible microorganisms.

Surprisingly, the combination of EDTA salt(s), Parabens and Methylene Blue has an unexpectedly enhanced rate of kill. The combination of EDTA salt(s), Parabens and Methylene Blue significantly reduces the reaction time necessary to kill a quantity of organisms.

### Experiment

The following materials were used (see Table 1):

TABLE 1

| Reagents/Chemicals | Manufacturer | Catalog # |
|---|---|---|
| Disodium EDTA (EDTA) | Fisher | S657-500 |
| Methyl Paraben (MP) | Sigma | H6654-100G |
| Propyl Paraben (PP) | Sigma | P5835-100G |
| Methylene Blue (MB) | Acros | 41424-0250 |
| MHB | Difco | 275730 |
| TSB | Difco | 211825 |

The following stock solutions to prepare required concentration:
For the EDTA stock solution, dissolved 12 g in 80 mls of Ion Pure DI water, adjusted pH to 8.5 using 1N HCl, and brought to volume with Ion Pure DI water;
For the Methylene Blue stock solution, dissolved 0.15 g in 80 mls of Ion Pure DI water, adjusted pH using 1N NaOH, and brought to volume with Ion Pure DI water; and
For the Paraben stock solution, dissolved 1.04 g Methyl Paraben and 0.12 g Propyl Paraben in 80 mls of Ion Pure DI water. Raised pH using 10N NaOH to dissolve parabens. Adjusted pH to 8.5 using 1N HCl and brought to volume with Ion Pure DI water.

The following concentrations were prepared:
For 50 mls of 4 wt % EDTA, 16.67 mls of the 12 wt % EDTA stock solution was diluted using 33.3 mls of Ion Pure DI water and mixed;
For 50 mls of 4 wt % EDTA+0.05 wt % MB, the 12 wt % EDTA stock solution was diluted to 8 wt % EDTA using 16.67 mls of Ion Pure DI water and 33.3 ml s of the stock solution. The 0.15 wt % MB stock solution was diluted to 0.1 wt % using 16.67 mls of Ion Pure DI water and 33.3 mls of the stock solution. In a new 50 mls test tube, equal parts of 8 wt % EDTA and 0.1 wt % MB were added and mixed;
For 50 mls of 4 wt % EDTA+0.04 wt % PP+0.345 wt % MP, the 8 wt % EDTA solution was used from above. The parabens stock solution was diluted to 0.69 wt % MP+0.08 wt % PP by diluting 33.3 mls of the parabens stock solution with 16.7 mls Ion Pure DI water. In a new test tube, equal parts of 8 wt % EDTA and 0.69 wt % MP+0.08 wt % PP were added and mixed; and
For 45 mls of 4 wt % EDTA+0.04 wt % PP+0.35 wt % MP+0.05 wt % MB, equal parts of the EDTA, parabens, and MB stock solutions were added to a 50 mls test tube and mixed.

The solutions stated in Table 2, below were subjected to antimicrobial testing.

TABLE 2

| No | Compound | Conc. (wt %/vol %) | pH |
|---|---|---|---|
| 1 | EDTA | 4 | 8.5 |
| 2 | EDTA + MB | 4 + 0.05 | 8.5 |
| 3 | EDTA + MP + PP | 4 + 0.345 + 0.04 | 8.5 |
| 4 | EDTA + MP + PP + MB | 4 + 0.345 + 0.04 + 0.05 | 8.5 |

A set of experiments was conducted to access if MP+PP, MB or MP+PP+MB significantly enhance EDTA antimicrobial activity against S. Aureus (ATCC-25923), S. Epidermidis (ATCC-12228), C. Albicans (ATCC-10231), P. Aeruginosa (ATCC-27853) via Rate of Kill Method.

The experiments followed method # TM-4339-82. Per this method, 4.9 mL of formulation was mixed with 0.1 mL of inoculum having 10⁷ (or the highest possible concentration) bacterial concentrations. The mixtures are then incubated at 37° C. for 0, 1, 3 & 24 hrs. After stipulated time the mixtures are removed, serially diluted and plated out. After 24 hrs plate count is obtained and is converted into log counts/recovery. Finally, the log reductions are calculated by taking the difference in log recovery between the formulations and the positive control.

The Rate of Kill results for S. Aureus (ATCC-25923) are shown in Table 3:

TABLE 3

Log Recovery

| Tested Compounds | T0 | T1 | T3 | T24 |
|---|---|---|---|---|
| EDTA | 6.6 | 6.6 | 6.5 | 4.6 |
| | 6.6 | 6.6 | 6.5 | 4.7 |
| Avg | 6.61 | 6.57 | 6.50 | 4.66 |
| EDTA + MB | 6.5 | 5.9 | 4.9 | 0 |
| | 6.6 | 6.2 | 5.0 | 0.0 |
| Avg | 6.56 | 6.04 | 4.94 | 0.00 |
| EDTA + PP + MP | 6.8 | 6.6 | 6.5 | 0 |
| | 6.8 | 6.7 | 6.6 | 0 |
| Avg | 6.80 | 6.61 | 6.54 | 0.00 |
| EDTA + PP + MP + MB | 6.1 | 0.0 | 0.0 | 0 |
| | 6.6 | 0.0 | 0.0 | 0 |
| Avg | 6.34 | 0.00 | 0.00 | 0.00 |
| +Cont. (10⁶) | 6.8 | 6.7 | 6.6 | 5.5 |
| | 6.4 | 6.6 | 6.7 | 5.7 |
| Avg | 6.57 | 6.65 | 6.66 | 5.62 |
| Neg. Cont. | 0.0 | 0.0 | 0.0 | 0 |

The data clearly show that:
EDTA+MB acts synergistically against S. Aureus (ATCC-25923);
EDTA+PP+MP acts synergistically against S. Aureus (ATCC-25923); and
EDTA+MB+PP+MP acts synergistically against S. Aureus (ATCC-25923) when compared with EDTA alone. This is because each of the formulations provides a ≧2 log reduction when compared with EDTA alone at 24 hr time point.

The Rate of Kill results for S. Epidermidis (ATCC-12228) are shown in Table 4:

TABLE 4

Log Recovery

| Tested Compounds | T0 | T1 | T3 | T24 |
|---|---|---|---|---|
| EDTA | 7.3 | 7.1 | 6.9 | 0.0 |
| | 7.2 | 7.2 | 6.9 | 0.0 |
| Avg | 7.24 | 7.14 | 6.89 | 0.00 |
| EDTA + MB | 7.1 | 6.2 | 0.0 | 0 |
| | 7.2 | 6.7 | 0.0 | 0.0 |
| Avg | 7.16 | 6.42 | 0.00 | 0.00 |
| EDTA + PP + MP | 7.3 | 7.2 | 6.6 | 0 |
| | 7.2 | 7.1 | 6.6 | 0 |
| Avg | 7.24 | 7.15 | 6.57 | 0.00 |
| EDTA + PP + MP + MB | 7.1 | 0.0 | 0.0 | 0 |
| | 7.2 | 0.0 | 0.0 | 0 |
| Avg | 7.16 | 0.00 | 0.00 | 0.00 |
| +Cont. (10⁶) | 7.3 | 7.3 | 7.0 | 6.1 |
| | 7.3 | 7.3 | 7.1 | 6.1 |
| Avg | 7.34 | 7.31 | 7.06 | 6.10 |
| Neg. Cont. | 0.0 | 0.0 | 0.0 | 0 |

The data clearly show that:
EDTA+MB acts synergistically against S. Epidermidis (ATCC-12228); and
EDTA+MB+PP+MP acts synergistically against S. Epidermidis (ATCC-12228) when compared with EDTA alone. This is because each of the formulations provides a ≧2 log reduction when compared with EDTA alone at 3 hr time point.

The Rate of Kill results for C. Albicans (ATCC-10231) are shown in Table 5:

TABLE 5

Log Recovery

| Tested Compounds | T0 | T1 | T3 | T24 |
|---|---|---|---|---|
| EDTA | 5.9 | 5.9 | 5.9 | 5.9 |
| | 6.1 | 5.9 | 5.9 | 5.9 |
| Avg | 6.00 | 5.89 | 5.89 | 5.91 |
| EDTA + MB | 6.0 | 5.8 | 4.7 | 0 |
| | 6.0 | 5.9 | 4.8 | 0.0 |
| Avg | 6.00 | 5.85 | 4.73 | 0.00 |
| EDTA + PP + MP | 6.0 | 5.9 | 6.0 | 5.98 |
| | 6.0 | 5.9 | 5.9 | 5.90 |
| Avg | 5.98 | 5.92 | 5.95 | 5.94 |
| EDTA + PP + MP + MB | 5.9 | 4.5 | 0.0 | 0.00 |
| | 5.9 | 4.6 | 0.0 | 0.00 |
| Avg | 5.89 | 4.55 | 0.00 | 0.00 |
| +Cont. (10⁶) | 5.9 | 5.8 | 5.9 | 6.0 |
| | 5.9 | 5.9 | 5.9 | 6.1 |
| Avg | 5.93 | 5.84 | 5.89 | 6.03 |
| Neg. Cont. | 0.0 | 0.0 | 0.0 | 0 |

The data clearly show that:
EDTA+MB acts synergistically against C. Albicans (ATCC-10231); and
EDTA+MB+PP+MP acts synergistically against C. Albicans (ATCC-10231) when compared with EDTA alone. This is because each of the formulations provides a ≧2 log reduction when compared with EDTA alone at 24 hr time point.

The Rate of Kill results for P. Aeruginosa (ATCC-27853) are shown in Table 6:

TABLE 6

Log Recovery

| Tested Compounds | T0 | T1 | T3 | T24 |
|---|---|---|---|---|
| EDTA | 5.8 | 4.6 | 3.0 | 0.0 |
| | 5.9 | 4.5 | 3.1 | 0.0 |
| Avg | 5.82 | 4.57 | 3.03 | 0.00 |
| EDTA + MB | 5.6 | 0.0 | 0.0 | 0 |
| | 5.5 | 0.0 | 0.0 | 0.0 |
| Avg | 5.57 | 0.00 | 0.00 | 0.00 |
| EDTA + PP + MP | 5.0 | 0.0 | 0.0 | 0 |
| | 4.8 | 0.0 | 0.0 | 0 |
| Avg | 4.91 | 0.00 | 0.00 | 0.00 |
| EDTA + PP + MP + MB | 2.2 | 0.0 | 0.0 | 0 |
| | 2.3 | 0.0 | 0.0 | 0 |
| Avg | 2.25 | 0.00 | 0.00 | 0.00 |
| +Cont. (10⁶) | 6.5 | 6.0 | 6.0 | 6.0 |
| | 6.5 | 6.0 | 5.9 | 6.1 |
| Avg | 6.52 | 6.00 | 5.95 | 6.06 |
| Neg. Cont. | 0.0 | 0.0 | 0.0 | 0 |

The data clearly show that:
EDTA+MB acts synergistically against P. Aeruginosa (ATCC-27853);
EDTA+PP+MP acts synergistically against P. Aeruginosa (ATCC-27853); and
EDTA+MB+PP+MP acts synergistically against P. Aeruginosa (ATCC-27853)
   when compared with EDTA alone. This is because each of the formulations provides a ≧2 log reduction when compared with EDTA alone at 1 hr time point.

The following table (Table 7) indicates what reagents acted synergistically with EDTA against various organisms.

TABLE 7

| | | | EDTA + | EDTA + |
|---|---|---|---|---|
| Organisms | EDTA | EDTA + MB | MP + PP | MP + PP + MB |
| S. Aureus | N/A | Yes | Yes | Yes |
| S. Epidermidis | N/A | Yes | No | Yes |
| C. Albicans | N/A | Yes | No | Yes |
| P. Aeruginosa | N/A | Yes | Yes | Yes. |

The data shows that solutions of EDTA salt(s), Parabens and Methylene Blue are synergistic. That is, embodiments of the combination of EDTA salt(s), Parabens and Methylene Blue provide results that are, unexpectedly, greater than the total effects of each agent operating by itself.

### Anticoagulant Experiments

Experiments were conducted to assess the anticoagulant activity of EDTA salt(s), Parabens and Methylene Blue, individually, and combinations of the three via a Prothrombin Time (PT) Assay. A PT assay (TM-4339-063) was conducted. Per the assay, the test samples are first mixed with normal anti-coagulated plasma in a ratio of 1:9 and incubated for 2 minutes at room temperature. Then tissue thromboplastin with calcium ions is added to the test samples. The samples are then incubated for 2 minutes at 37° C. When tissue thromboplastin and calcium ions is added to normal anti-coagulated plasma mixed with formulation, the clotting mechanism is initiated leading to formation of a fibrin clot. The Coagulation Analyzer is utilized to record the PT required for clot formation. The PT so obtained is then converted to International Normalized Ratio (INR).

Tetrasodium EDTA (TEDTA) was used (Alfa Aesar, Catalog #A17385). TriniCHECK 1 (Normal Control) was used (Trinity Biotech). TriniCHECK 2 & 3 (Abnormal Controls) were used (Trinity Biotech). A KC4 Amelung Coagulizer was used (Trinity Biotech).

FIG. 5 shows the results of the PT assay. The values stated in the “Conc. wt %” column are the final concentrations of the reagents. TriniCHECK Level 1 is a lyophilized human plasma with characteristics similar to those of fresh normal plasma to be used as a normal control in the Prothrombin Time (PT), assay procedures. TriniCHECK Level 2 and TriniCHECK Level 3 are lyophilized human plasmas in which Factors II, VII, IX, and X have been selectively and partially removed, to be used as abnormal controls in the Prothrombin Time (PT) test procedures. INR (International Normalized Ratio) is a system established by the World Health Organization (WHO) and the International Committee on Thrombosis and Hemostasis for reporting the results of blood coagulation (clotting) tests. INR is calculated as:

INR=(PTtest sample/PTnormal control)ISI

ISI (International Sensitivity Index) indicates the sensitivity of individual thromboplastin. The value of ISI utilized herein was 1.65.

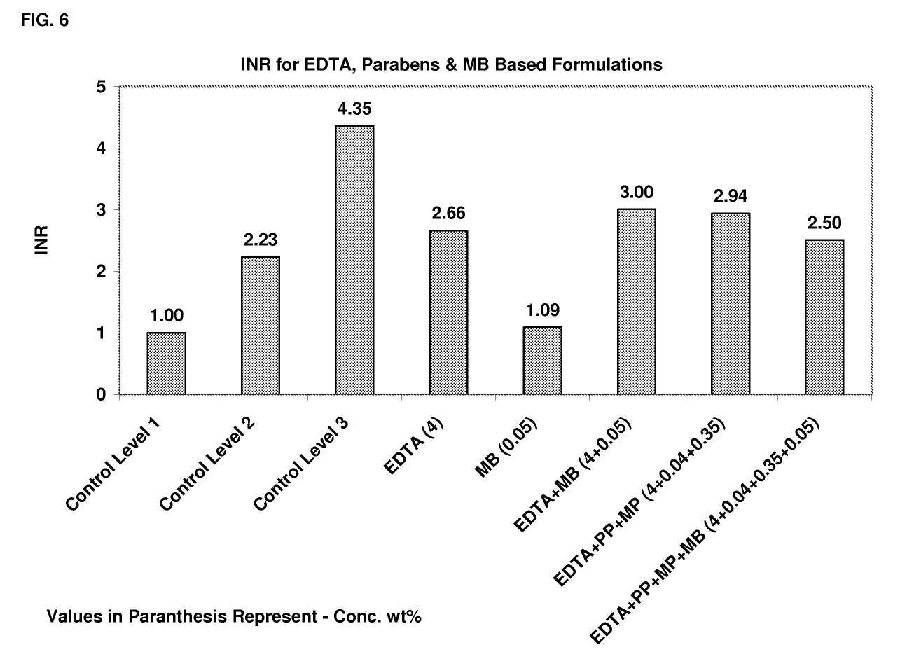

FIG. 6 shows the graph of the International Normalized Ratio (INR) for EDTA, MB, EDTA+MB, EDTA+PP+MP and EDTA+PP+MP+MB from a Prothrombin Time (PT) Assay.

From FIG. 6 it is evident that (within the tested range) EDTA (4 wt %) mixed with 0.05 wt % MB provides anticoagulant activity 34% greater than the Control Level 2.

From FIG. 6 it is evident that (within the tested range) EDTA (4 wt %) mixed with 0.04 wt % PP & 0.35 wt % MP provides anticoagulant activity 31.5% greater than the Control Level 2.

From FIG. 6 it is evident that (within the tested range) MB+PP+MP neither significantly promote or enhance the anticoagulant activity of EDTA, nor does negatively affect the anticoagulant activity of EDTA. In addition, for the combination formulation the INR values are slightly greater than the Control Level 2, thus indicating the anticoagulant efficacy of the formulation.

From the foregoing, it should be clear that the present disclosure may be embodied in forms other than those discussed above; the scope of the present disclosure should be determined by the following claims and not the detailed discussion presented above.

1. A disinfectant composition comprising:
at least one EDTA salt;
at least one paraben of the following formula: 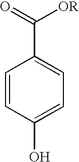
where R symbolizes an alkyl group; and
methylene blue,
in solution, wherein the at least one EDTA salt is at a concentration of at least 0.01°/o (w/v) and less than 20.0% (w/v), the at least one paraben is at a concentration of 0.005% (w/v) and less than 1% (w/v), and the methylene blue is at a concentration of 0.001% (w/v) and less than 1% (w/v) and the disinfectant composition has a pH greater than 7.5.

2. The disinfectant composition of claim 1, wherein the disinfectant composition has a pH from 8.0 to 12.0.

3. The disinfectant composition of claim 1, wherein the disinfectant composition has a pH from 8.0 to 9.5.

4. The disinfectant composition of claim 1, wherein the disinfectant composition has a pH from 8.0 to 9.0.

5. The disinfectant composition of claim 1, wherein the at least one EDTA salt is a sodium salt.

6. The disinfectant composition of claim 1, wherein the at least one paraben comprises methyl paraben and propyl paraben.

7. The composition of claim 1, wherein the solution comprises between 0 and 10% (v/v) ethanol and water.

8. The composition of claim 1, wherein the solution comprises saline.

9. The composition of claim 1, wherein the at least one EDTA salt is at a concentration of at least 0.25% (w/v) and less than 12.0% (w/v), the at least one paraben is at a concentration of 0.01% (w/v) and less than 1% (w/v), and the methylene blue is at a concentration of 0.01% (w/v) and less than 0.5% (w/v).

10. The composition of claim 1, wherein the disinfectant composition is packaged in a sterile, non-pyrogenic form, the solution is water, and the disinfectant composition has an osmolarity of from 240-500 mOsM/Kg.

11. A disinfectant composition comprising:
at least one EDTA salt;
at least one paraben of the following formula: 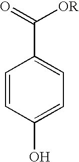
where R symbolizes an alkyl group; and
methylene blue,
in a dry or partially hydrated formulation that, upon reconstitution with a solution, forms the disinfectant composition of claim 1.

12. A lock flush composition comprising:
at least one EDTA salt;
at least one paraben of the following formula: 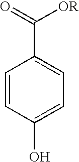
where R symbolizes an alkyl group; and
methylene blue,
in solution, wherein the at least one EDTA salt is at a concentration of at least 0.01°/o (w/v) and less than 20.0% (w/v), the at least one paraben is at a concentration of 0.005% (w/v) and less than 1% (w/v), and the methylene blue is at a concentration of 0.001% (w/v) and less than 1% (w/v), and the disinfectant composition has a pH greater than 7.5, the lock flush composition is packaged in a sterile, non-pyrogenic form, and the lock flush composition is biocompatible for use in in-dwelling access catheters, urinary catheters, nasal tubes and throat tubes.

13. The composition of claim 12, wherein the at least one EDTA salt is at a concentration of at least 0.25% (w/v) and less than 12.0% (w/v), the at least one paraben is at a concentration of 0.01% (w/v) and less than 1% (w/v), and the methylene blue is at a concentration of 0.01% (w/v) and less than 0.5% (w/v).

14. A method for disinfecting a surface by contacting the surface with a disinfectant solution comprising:
at least one EDTA salt;
at least one paraben of the following formula: 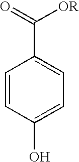
where R symbolizes an alkyl group; and
methylene blue,
in solution, wherein the at least one EDTA salt is at a concentration of at least 0.01°/o (w/v) and less than 20.0% (w/v), the at least one paraben is at a concentration of 0.005% (w/v) and less than 1% (w/v), and the methylene blue is at a concentration of 0.001% (w/v) and less than 1% (w/v) and the disinfectant composition has a pH greater than 7.5.

15. The method of claim 14, wherein the solvent is water.

16. The method of claim 14, wherein the at least one EDTA salt is at a concentration of at least 0.25% (w/v) and less than 12.0% (w/v), the at least one paraben is at a concentration of 0.01% (w/v) and less than 1% (w/v), and the methylene blue is at a concentration of 0.01% (w/v) and less than 0.5% (w/v).

17. The method of claim 14, wherein the surface of a medical device.

18. The method of claim 14, wherein the surface is a conduit.

19. The method of claim 14, wherein the surface is a catheter and contacting the catheter with the disinfectant solution is accomplished by locking, flushing or coating the catheter with the disinfectant solution.

20. The method of claim 14, wherein the catheter is selected from the group consisting of peripherally inserted catheters, central venous catheters, peritoneal catheters, hemodialysis catheters and urological catheters.

21. The method of claim 19, wherein contacting the catheter comprises:
introducing a disinfectant solution into an interior lumen of the catheter;
holding the disinfectant solution within the lumen for a selected period of time; and
removing the disinfectant solution from the interior lumen.

22. An anticoagulant composition comprising:
at least one EDTA salt;
at least one paraben of the following formula: 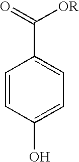
where R symbolizes an alkyl group; and
methylene blue,
in solution, wherein the at least one EDTA salt is at a concentration of at least 0.01% (w/v) and less than 20.0% (w/v), the at least one paraben is at a concentration of 0.005% (w/v) and less than 1% (w/v), and the methylene blue is at a concentration of 0.001% (w/v) and less than 1% (w/v) and the disinfectant composition has a pH greater than 7.5.

23. The composition of claim 22, wherein the at least one EDTA salt is at a concentration of at least 0.25% (w/v) and less than 12.0% (w/v), the at least one paraben is at a concentration of 0.01% (w/v) and less than 1% (w/v), and the methylene blue is at a concentration of 0.01% (w/v) and less than 0.5% (w/v).
